SEQUENCE LISTING (2 sequences)

SEQ ID NO: 1 - protein, 543 aa, synthetic construct
MRSNVLTVYSPYQSNLIRPILNEFEKQEHVKIEIKHGSTQVLLSNLHNEDFSERGDVFMGGVLSETIDHPEDFVPYQDTSVTQQLEDYRSNNKYVTSFLLMPTVIVVNSDLQGDIKIRGYQDLLQPILKGKIAYSNPNTTTTGYQHMRAIYSMHHRVSDVHQFQNHAMQLSKTSKVIEDVAKGKYYAGLSYEQDARTWKNKGYPVSIVYPIEGTMLNVDGIALVKNAHPHPKRKKLVQYLTSRSVQQRLVAEFDAKSIRKDVSEQSDLANVYIMADKQKNGIKANFKIRHNVEDGSVQLADHYQQNTPIGDGPVLLPDNHYLLTQSVISKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNLCQIIENLKNIPLIPKSKLPDIPHHKFLEMIQLQ

SEQ ID NO: 2 - protein, 545 aa, synthetic construct
MRSNVLTVYSPYQSNLIRPILNEFEKQEHVKIEIKHGSTQVLLSNLHNEDFSERGDVFMGGVLSETIDHPEDFVPYQDTSVTQQLEDYRSNNKYVTSFLLMPTVIVVNSDLQGDIKIRGYQDLLQPILKGKIAYSNPNTTTTGYQHMRAIYSMHHRVSDVHQFQNHAMQLSKTSKVIEDVAKGKYYAGLSYEQDARTWKNKGYPVSIVYPIEGTMLNVDGIALVKNAHPHPKRKKLVQYLTSRSVQQRLVAEFDAKSIRKDVSEQSDQSIENLRLSHNVYIMADKQKNGIKANFKIRHNVEDGSVQLADHYQQNTPIGDGPVLLPDNHYLNTQSVLSKDPNEKRDHMVLLEFVTAAGITLGMDELYKGGTGGSMSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNQTIPLIPKSKLPDIPHHKFLEMIQLQ

(19) United States
(12) Patent Application Publication
(10) Pub. No.: US 2026/0295095 A1
(43) Pub. Date: Oct. 1, 2026

(54) FLUORESCENT SENSORS AND METHODS OF MAKING AND USING

(71) Applicant: Howard Hughes Medical Institute, Chevy Chase, MD (US)

(72) Inventor: Jonathan S. Marvin, Arlington, VA (US)

(21) Appl. No.: 19/636,395

(22) Filed: Apr. 1, 2026

Related U.S. Application Data
(60) Provisional application No. 63/781,417, filed on Apr. 1, 2025.

(51) Int. Cl. A61K 49/00; C07K 14/00

(52) U.S. Cl. CPC A61K 49/0045; C07K 14/001

(57) ABSTRACT

This disclosure describes fluorescence-based sensors for detecting and quantifying glucose-6-phosphate (G6P), and methods of using such sensors in vitro and in vivo.

### CROSS REFERENCE TO RELATED APPLICATIONS

This application claims the benefit of priority under 35 U.S.C. § 119 (e) to U.S. Application No. 63/781,417 filed Apr. 1, 2025, which is incorporated by reference herein in its entirety.

### SEQUENCE LISTING

This application contains a Sequence Listing that has been submitted electronically as an XML file named “30872-0035001.XML.” The XML file, created on May 11, 2026, is 3,856 bytes in size. The material in the XML file is hereby incorporated by reference in its entirety.

### TECHNICAL FIELD

This disclosure generally relates to fluorescent sensors and methods of making and using such fluorescent sensors.

### BACKGROUND

Glucose-6-phosphate (G6P) plays a central role in cellular metabolism and is a critical intermediate in pathways such as glycolysis, gluconeogenesis, and the pentose phosphate pathway. Its regulation is vital for maintaining cellular energy homeostasis and proper metabolic function.

### SUMMARY

Fluorescence-based sensors for detecting and quantifying G6P are described herein, as are methods of making and using such sensors. Here we report the development of an intensity-based G6P sensing fluorescent reporter, iG6PSnFR. iG6PSnFR can be fused to spectrally separable fluorescent tags (HaloTag with synthetic far-red fluorophores or mIRFP670nano3), thus providing an approach to normalize the signal to the expression level of the sensor and allowing quantitative comparisons across individual cells or subcellular locations. We show that iG6PSnFR can provide detailed measurements of the variations in G6P levels as well as the kinetics of changes in G6P.

In one aspect, G6P-detecting fluorescence-based sensors are provided that have at least 95% sequence identity (e.g., at least 96% sequence identity, at least 97% sequence identity, at least 98% sequence identity, at least 99% sequence identity, 100% sequence identity) to an amino acid sequence selected from SEQ ID NOs: 1 and 2.

In another aspect, a nucleic acid encoding any of the G6P-detecting fluorescence-based sensors described herein. In still another aspect, vectors are provided that include any of the nucleic acids described herein. In yet another aspect, cells are provided that include any of the nucleic acids described herein or any of the vectors described herein.

In one aspect, methods of determining the amount of G6P in a cell are provided. Such methods typically include providing a cell comprising any of the G6P-detecting fluorescence-based sensors described herein and determining the amount of fluorescence emitted from the sensor.

In some embodiments, such methods further include exposing the cell to G6P, an inhibitor of G6P, or another biochemical stimuli. In some embodiments, the cell expresses any of the nucleic acids described herein. In some embodiments, the cell includes any of the vectors described herein. In some embodiments, the cell is in vitro. In some embodiments, the cell is in vivo. In some embodiments, the cell is from an organism that suffers from a metabolic disorder (e.g., diabetes).

In still another aspect, articles of manufacture are provided that include any of the G6P-detecting fluorescence-based sensors described herein, any of the nucleic acids described herein, or any of the vectors described herein. In some embodiments, the articles of manufacture further include G6P. In some embodiments, the articles of manufacture further include a G6P inhibitor. In some embodiments, the articles of manufacture further include a biochemical stimuli. In some embodiments, the articles of manufacture further include instructions for using the G6P-detecting fluorescence-based sensor.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

The patent or application file contains at least one drawing executed in color. Copies of this patent or patent application publication with color drawings will be provided by the Office upon request and payment of the necessary fee.

FIG. 1A-1B are graphs showing the titration of iG6PSnFR sensors 109D9 (FIG. 1A, red) and 66C5 (FIG. 1B, pink) with G6P in TBS. Kds are 10 μM and 1 μM, respectively.

FIG. 2A-2B are graphs showing that iG6PSnFR (109D9) does not respond significantly to fructose-6-phosphate nor glucose-1-phosphate (FIG. 2B, blue or grey), and the iG6PSnFR (109D9) affinity for G6P (FIG. 2B, red) is not affected by 10 mM glucose (FIG. 2B, grey).

FIG. 3A-3B are graphs showing that iG6PSnFR detects glucose-6-phosphate (black) but not 2-deoxyglucose-6-phosphate (medium grey) nor 6-phosphogluconate (light grey). FIG. 3A is sensor 109D9; FIG. 3B is sensor 66C5. The slight drop in fluorescence at high concentrations of 2-deoxyglucose-6-phosphate is an artefact of the stock solution being at a pH of about 6.5.

FIG. 4A-4B are graphs showing the titration of iG6PSnFR with G6P at different concentrations of inorganic phosphate. FIG. 4A is sensor 109D9; FIG. 4B is sensor 66C5. Buffer is Tris Buffered Saline, with PO4 concentrations of 0 mM (blue), 2 mM (green), 10 mM (yellow), 50 mM (orange), 100 mM (red).

FIG. 5 is a graph demonstrating that HeLa cells equilibrated with variable concentrations of glucose (0 mM, 0.125 mM, 0.25 mM, 0.5 mM, 1 mM, and 2 mM, from lightest to darkest blue) show increased fluorescence of iG6PSnFR.109D9, indicating higher cytosolic G6P, as expected. Treatment with 2-deoxyglucose, which blocks conversion of glucose to G6P by hexokinase, resulted in a rapid drop in iG6PSnFR.109D9 fluorescence, as expected.

FIG. 6A-6B are graphs showing that HeLa cells expressing iG6PSnFR.109D9 (FIG. 6A) or iG6PSnFR.66C5 (FIG. 6B) equilibrated with 0.25 mM glucose and then treated with 10 μM Glutor (blue) to inhibit the GLUT glucose transporters or with 5 mM 2-deoxyglucose (orange) to inhibit conversion of glucose to G6P by hexokinase. Treatment with no drug (grey) results in no change in fluorescence.

FIG. 7A-7B are graphs showing HeLa cells expressing iG6PSnFR. 109D9 (FIG. 7A) or iGlucoSnFR2 (FIG. 7B) equilibrated with 1 mM glucose and treated with 1 mM, 2 mM, or 5 mM glucose (light, medium, and dark grey). Cytosolic G6P levels do not change with those treatments (FIG. 7A) but glucose concentrations do (FIG. 7B). Treatment with 10 UM Glutor is shown in blue; treatment with 100 μM Lonidamine is shown in purple; and treatment with 5 mM pyruvate is shown in red.

FIG. 8 is a plot of the expression of the 109D9 variant of iG6PSnFR.HaloTag coupled with JF635 in cultured neurons. At the times indicated about the graph, the media was exchanged for media containing 0 mM glucose and fluorescence dropped after a short lag. Fluorescence from the iG6PSnFR sensor was restored by adding 5 mM glucose back to the media.

FIG. 9 is a plot showing the expression of iG6PSnFR in cultured neurons stimulated with 100 field stimuli every 50 msec.

FIG. 10 is a plot showing four different metabolites (glucose-6-phosphate (G6P), glucose, fructose 1,6-bisphosphate (FBP), and pyruvate) in parallel experiments in cultured neurons in response to electrical stimulation.

FIG. 11A shows the intrinsic fluorescence of NADH recorded in the blue channel (Ex 340 nm, em 460 nm), which is often used to report glycolysis in cells. When glycolysis is high, NAD+ is reduced to NADH and fluorescence at 460 nm increases. The fluorescence of the 66C5 variant of iG6PSnFR was recorded in the green channel (Ex 475 em 515 nm) and was used to report changes in G6P concentration.

FIG. 11B shows FuraRed, a calcium sensitive fluorescent reporter that has a broad excitation spectrum and emits at 640 nm, used for comparison purposes.

FIGS. 12A-12C describe intravital imaging of iG6PSnFR. FIG. 12A is a schematic showing the experimental setup illustrating how the liver is imaged by microscopy and how proximity to the portal or central veins dictate the balance of gluconeogenesis and glycolysis. FIG. 12B shows images of “normal” sensor 109D9 in green and mIRFP670nano3 in red showing more G6P (more green) near the central vein. A heat map is shown on the right. FIG. 12C shows images of “inverse” sensor 66C5 also demonstrating more G6P (less green) near the central vein. A heat map is shown on the right.

### DETAILED DESCRIPTION

Due to the significant role that glucose-6-phosphate (G6P) plays in cellular metabolism and energy homeostasis as well as glycolysis, gluconeogenesis, and the pentose phosphate pathway, precise measurement of G6P concentration is essential for understanding metabolic disorders (e.g., diabetes) and for monitoring cellular responses to various biochemical stimuli.

Traditional methods for detecting G6P, including enzymatic assays and chromatography-based techniques (see, e.g., Bassi et al., 1999, Anal. Biochem., 268 (2): 223-8, which describes the conversion of NAD+ to NADH, the latter of which has a higher absorbance at 340 nm), often involve complex sample preparation, long analysis times, and require expensive instrumentation. Even the available G6P biosensors are complicated (see, e.g., Kim et al., 2024, Sensors and Actuators B: Chemical, 418:136272, which describes a multicomponent electrochemical sensor for G6P has been constructed from an electrode, nanomesh composed from M13 phage, a polyelectrolyte, and E. coli displaying G6P dehydrogenase). These limitations make high-throughput or real-time monitoring of G6P challenging, particularly in biological systems where fast and non-invasive detection is desirable.

Fluorescence-based sensors offer a promising alternative, providing advantages such as high sensitivity, rapid response times, and the potential for real-time monitoring in both in vitro and in vivo settings. Here we describe the design, optimization, and application of a novel fluorescence sensor for the detection of glucose-6-phosphate, with potential applications including basic metabolic research, drug discovery, and potentially clinical diagnostics.

Successful deployment of a fluorescent biosensor requires that the affinity of the sensor matches the physiological concentration of its cognate analyte and that the sensor discriminates against chemically related species. The signal-to-noise ratio is determined by the ratio of the sensor's fluorescence in the bound and unbound states and sets the limit of what magnitude changes in the analyte concentration can be detected in the face of other sources of fluctuation and, therefore, the spatial scale over which the signal must be averaged.

Here, we present novel G6P sensors referred to as iG6PSnFR. We validated their utility for measuring changes in G6P using cellular imaging in cultured cell lines, perturbed with different pharmacological compounds known to affect G6P production and consumption. Inclusion of a far-red fluorophore (via HaloTag or mIRFP670nano3) allows for ratiometric measurement to account for expression and movement artefacts. An iG6PSnFR sensor can be targeted to any part of the cell (e.g., nucleus, mitochondria, endoplasmic reticulum), where it reports homeostasis of G6P. It also reports depletion and subsequent recovery of G6P.

We expect that iG6PSnFR will provide researchers with previously exciting new opportunities to study G6P dynamics with temporal and spatial resolution that has, until now, been unavailable.

iG6PSnFR Sequences
iG6PSnFR.109D9(mutations are underlined:
cpSFGFP.S147L, cpSFGFP.H148A, cpSFGFP.Y180D,
cpSFGFP.S202L, cpSFGFPL207I, cpSFGFP.F145L,
cpSFGFP.N146C, 6LKK.S294I)
(SEQ ID NO: 1)
MRSNVLTVYSPYQSNLIRPILNEFEKQEHVKIEIKHGSTQVLLSNLHNED
FSERGDVFMGGVLSETIDHPEDFVPYQDTSVTQQLEDYRSNNKYVTSFLL
MPTVIVVNSDLQGDIKIRGYQDLLQPILKGKIAYSNPNTTTTGYQHMRAI
YSMHHRVSDVHQFQNHAMQLSKTSKVIEDVAKGKYYAGLSYEQDARTWKN
KGYPVSIVYPIEGTMLNVDGIALVKNAHPHPKRKKLVQYLTSRSVQQRIV
AEFDAKSIRKDVSEQSDLANVYIMADKQKNGIKANEKIRHNVEDGSVQLA
DHYQQNTPIGDGPVLLPDNEYLLTQSVISKDPNEKRLHMVLLEFVTAAGI
TLGMDELYKGGTGGSMSKGEELFTGVVPILVELDGDVNGHKFSVRGEGEG
DATNGKLTLKFICTTGKLPVPWPTLYTTLTYGVQCFSRYPDHMKQHDFFK
SAMPEGYVQERTISFKDDGTYKTRAEVKFEGDTLVNRIELKGIDFKEDGN
ILGHKLEYNLCQIIENLKNIPLIPKSKLPDIPHHKFLEMIQLQ
iG6PSnFR.66C5 (insertion is underlined:
RL between 6LKK.L298 and cpSFGFP.S147;
mutations are underlined: cpSFGFP.Y180D, cpSFGFP.
S202N, 6LKK.K299Q, 6LKK.N300T)
(SEQ ID NO: 2)
MRSNVLTVYSPYQSNLIRPILNEFEKQEHVKIEIKHGSTQVLLSNLHNED
FSERGDVFMGGVLSETIDHPEDFVPYQDTSVTQQLEDYRSNNKYVTSFLL
MPTVIVVNSDLQGDIKIRGYQDLLQPILKGKIAYSNPNTTTIGYQHMRAI
YSMHHRVSDVHQFQNHAMQLSKTSKVIEDVAKGKYYAGLSYEQDARTWKN
KGYPVSIVYPIEGTMLNVDGIALVKNAHPHPKRKKLVQYLTSRSVQQRLV
AEFDAKSIRKDVSEQSDQSIENLRLSHNVYIMADKQKNGIKANFKIRHNV
EDGSVQLADHYQQNTPIGDGEVLLPDNHYLNTQSVLSKDPNEKRDHMVLL
EFVTAAGITLGMDELYKGGTGGSMSKGEELFTGVVPILVELDGDVNGHKF
SVRGEGEGDATNGKLTLKFICTTGKLPVPWPTLVTTLTYGVQCFSRYPDH
MKQHDFFKSAMPEGYVQERTISFKDDGTYKTRAEVKFEGDTLVNRIELKG
IDFKEDGNILGHKLEYNFNQTIPLIPKSKLPDIPHHKFLEMIQLQ

Black=restriction sites for subcloning (RS=BglII, LQ=PstI)

Purple=HptA (Numbered according to 6I.KK.pdb)

Green=circulary premured SUPERFOLDERgfp (numbered according to standard GFP numbering, with the chromophore formed from residues 65.66,67 (TYG)

Red=residues that were inserted between the binding protein and the cpSFGFP

It would be appreciated that another protein can be added to the C-Terminus of the sensor for normalization of expression and focus artefacts. That protein can be another fluorescent protein, that is either pH-insensitive (such as mRuby3) or pH-sensitive (such as pH-mScarlet) or it can be a protein that can be specifically labeled with synthetic fluorophores, such as HaloTag or SnapTag. The synthetic fluorophores can be pH-insensitive (such as JF-X650) or pH-sensitive (such as LAMP-shade violet).

In addition to the polypeptide sensors disclosed herein (i.e., SEQ ID NOs: 1 and 2), the skilled artisan will further appreciate that changes can be introduced into a nucleic acid molecule encoding a sensor, thereby leading to changes in the amino acid sequence of the encoded polypeptide (e.g., SEQ ID NOs: 1 and 2). For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5 (Suppl. 3): 345-352), which provides frequency tables for amino acid substitutions), and a non-conservative substitution is one in which an amino acid residue is replaced with an amino acid residue that does not have a similar side chain.

Also provided are polypeptides that differ from SEQ ID NOs: 1 and 2. Polypeptides that differ in sequence from SEQ ID NOs: 1 and 2 can have at least 95% sequence identity (e.g., at least 96%, 97%, 98%, 99% or 100% sequence identity) to SEQ ID NOs: 1 and 2. In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

As used herein, an “isolated” nucleic acid molecule is a nucleic acid that is separated from other nucleic acids that are usually associated with the reference nucleic acid in the genome. Thus, an “isolated” nucleic acid includes, without limitation, a nucleic acid that is free of sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid is generally introduced into a vector (e.g., a cloning vector, or an expression vector) for convenience of manipulation or to generate a fusion nucleic acid molecule. In addition, an isolated nucleic acid can include an engineered nucleic acid molecule such as a recombinant or a synthetic nucleic acid.

Isolated nucleic acids can be obtained using techniques routine in the art. For example, isolated nucleic acids can be obtained using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Vectors containing nucleic acids that encode polypeptides also are provided. Vectors, including expression vectors, suitable for use are commercially available and/or produced by recombinant DNA technology methods routine in the art. A vector containing a nucleic acid can have elements necessary for expression operably linked to such a nucleic acid, and further can include sequences such as those encoding a selectable marker (e.g., an antibiotic resistance gene), and/or those that can be used in purification of a polypeptide (e.g., 6×His tag).

Elements necessary for expression include nucleic acid sequences that direct and regulate expression of nucleic acid coding sequences. One example of an element necessary for expression is a promoter sequence. Elements necessary for expression also can include introns, enhancer sequences, response elements, or inducible elements that modulate expression of a nucleic acid. Elements necessary for expression can be of bacterial, yeast, insect, mammalian, or viral origin and vectors can contain a combination of elements from different origins. Elements necessary for expression are described, for example, in Goeddel, 1990, Gene Expression Technology: Methods in Enzymology, 185, Academic Press, San Diego, CA. As used herein, operably linked means that a promoter and/or other regulatory element(s) are positioned in a vector relative to a nucleic acid in such a way as to direct or regulate expression of the nucleic acid.

Another aspect pertains to host cells into which a vector, e.g., an expression vector, or an isolated nucleic acid molecule has been introduced. Many methods for introducing nucleic acids into host cells, both in vivo and in vitro, are well known to those skilled in the art and include, without limitation, calcium phosphate precipitation, electroporation, heat shock, lipofection, microinjection, and viral-mediated nucleic acid transfer. The term “host cell” refers not only to the particular cell but also to the progeny or potential progeny of such a cell. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

The term “purified” polypeptide as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide is “purified.”

Polypeptides can be purified from natural sources (e.g., a biological sample) by known methods such as DEAE ion exchange, gel filtration, and hydroxyapatite chromatography. A purified polypeptide also can be obtained, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

This disclosure also provides for articles of manufacture that can be used to detect G6P. An article of manufacture as provided herein can include any of the G6P-detecting fluorescence-based sensor described herein (e.g., nucleic acids, polypeptides, vectors and/or host cells) together with suitable packaging materials.

Articles of manufacture provided herein also can contain a package insert or package label having instructions thereon for using any of the G6P-detecting fluorescence-based sensor described herein to determine the presence and/or amount of G6P in one or more cells. Articles of manufacture may additionally include reagents for carrying out the methods disclosed herein (e.g., buffers, enzymes, or co-factors).

In accordance with the present disclosure, there may be employed molecular biology, microbiology, biochemical, and recombinant DNA techniques within the skill of the art. Such techniques are explained fully in the literature. The compositions and methods will be further described in the following examples, which do not limit the scope of the matter described in the claims.

### EXAMPLES

### Example 1—Development of a Sensor and In Vitro Characterization

We first intended to develop a sensor for G6P by redesigning our existing sensor for glucose, iGlucoSnFR2. We browsed the protein structure database to find structures that bind G6P that could guide our attempts to redesign the ligand binding pocket and came across two structures that bind G6P: a G6P-associated sensor protein (HptA) from Staph aureus (Wang et al., 2020, PNAS USA, 117 (48): 30433-40) and a G6P transporter from Actinobacillus pleuropneumoniae (AfuA) (Sit et al., 2015, PLOS Pathog., 11 (8): e1005107). Both are reported to be periplasmic binding proteins with Venus-flytrap mechanisms for binding their ligands, but the former is reported to only bind G6P and galactose-6-phosphate, while the latter is reported to also bind fructose-6-phosphate. As such, we went forward with plans to turn HptA (6LKK.pdb) into an intensity-based sensor.

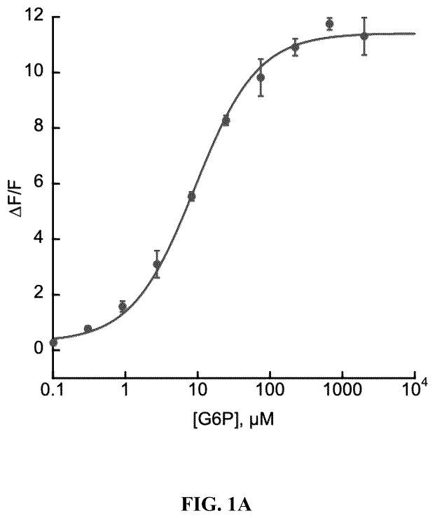

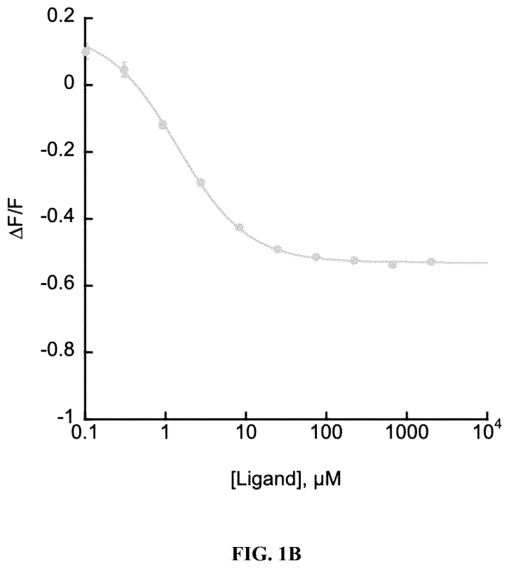

Based on our previous successes in making fluorescence sensors from Venus-flytrap like proteins and circularly permuted fluorescent proteins, we chose four positions into which we would insert cpSFGFP: after residues 93, 196, 292, or 298 (numbering based on 6LKK.PDB). We then screened ~400 variants in four groups for each insertion position. For the first binding-protein junction, we randomized the last two residues of the binding protein or the first two residues of cpSFGFP (ie, . . . . SHNVY . . . mutated to . . . XXNVY . . . ). For the second junction, the last two residues of cpSFGFP were randomized ( . . . FNYN . . . mutated to . . . FNXX . . . ) or the first two residues of the binding protein. Insertions at residues 93, 292, and 298 produced sensors, while insertion of cpSFGFP after residue 196 of 6LKK did not. With higher ΔF/F sensors resulting from positions 292 and 298, we carried them forward with additional screening. We ended up identifying three sensors that we deemed suitable for testing in cell culture. 109D9 (with cpSFGFP inserted after residue 292 of 6LKK) has a maximum ΔF/F of ~12 as purified protein (FIG. 1A) and a Kd of 10 μM when assayed in Tris Buffered Saline (50 mM Tris, 150 mM NaCl, pH 7.5). Variants 66C5 and 68G10 (both with cpSFGFP inserted after residue 298 of 6LKK) are inverse sensors with maximum ΔF/F of negative 0.65 (FIG. 1B) and Kd of 1 and 10 μM, respectively, in TBS.

### Example 2—Ligand Binding Specificity

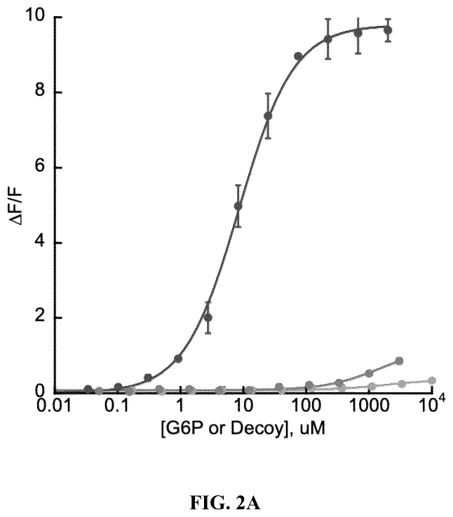

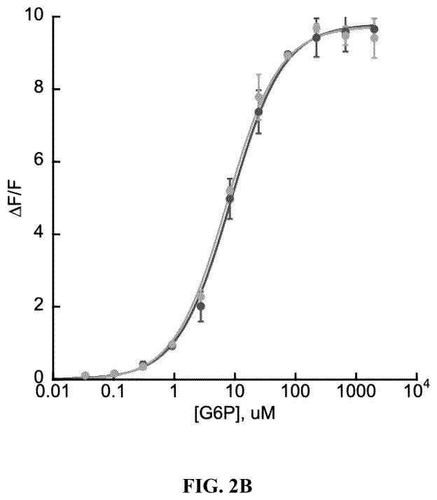

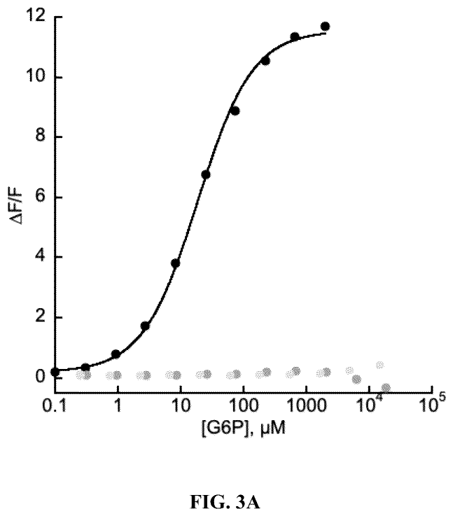

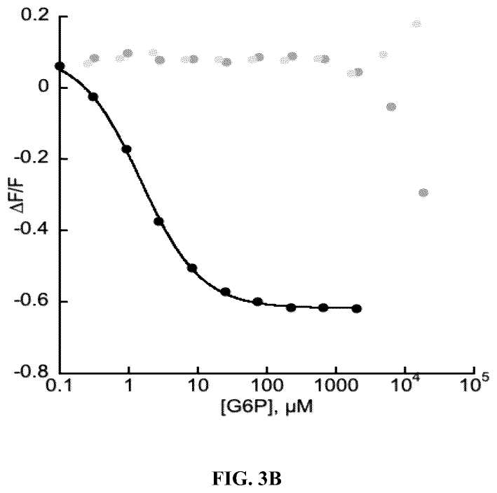

A critical aspect of biosensors is their ligand binding specificity. HptA was reported to be highly specific for G6P as determined by isothermal titration calorimetry (ITC) (Wang et al., 2020, PNAS USA, 117 (48): 30433-40). However, ITC is limited in its ability to detect weak (i.e., 100 micromolar to low millimolar) binding, or competitive inhibition. Thus, we tested a panel of relevant decoys: glucose, glucose-1-phosphate, fructose-6-phosphate, fructose-1,6-bisphosphate, 2-deoxyglucose and 2-deoxyglucose-6-phoshate, and inorganic phosphate. The sensor does not change fluorescence in response to any of these compounds (FIGS. 2A, 3A and 3B). In addition, the sensor's affinity for G6P is not affected by 10 mM glucose (FIG. 2B, the compound most likely to be present at high concentrations in cells.

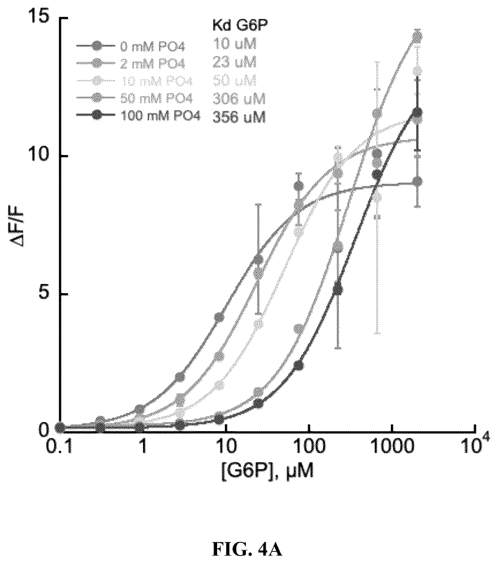

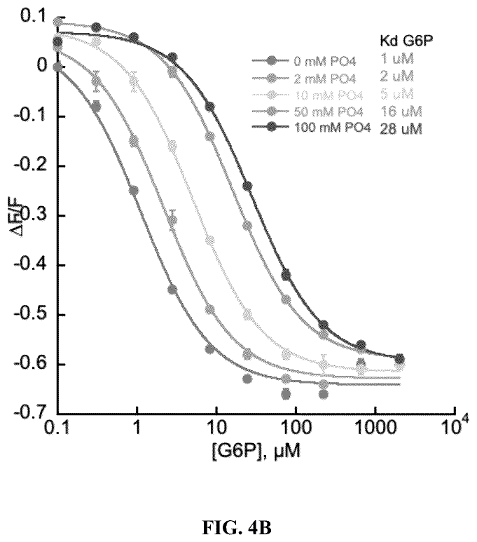

However, the sensor's affinity for G6P is affected by inorganic phosphate. The affinity of G6P sensor 109D9 for G6P decreases about 30-fold from 10 μM in the absence of PO4 to 350 μM in the presence of 100 mM PO4 (FIG. 4A), as its maximum ΔF/F increase from ~10 to ~15. The G6P affinity of the inverse sensor 66C5 also decreases about 30-fold, from 1 μM in the absence of PO4 to 28 μM in the presence of 100 mM PO4, but the maximum ΔF/F is relatively the same (FIG. 4B). This effect of PO4 on the sensor's affinity for G6P means that an increase in fluorescence of the 109D9 sensor could result from micromolar increases in G6P concentration (i.e., the sensor detecting A [G6P] as expected and desired) or possibly from millimolar decreases in PO4 concentration).

Both iG6PSnFR sensor variants have a small change in their fluorescence lifetimes between the unbound and bound states. For 109D9, the single exponential lifetime decreases from 2.32 nsec to 2.12 nsec (SD is +0.03 nsec for both). For sensor 66C5, the single exponential lifetime decreases from 2.44 nsec to 2.28 nsec (SD is =0.01 nsec for both).

### Example 3—Validation of iG6PSnFRs in Mammalian Cell Culture

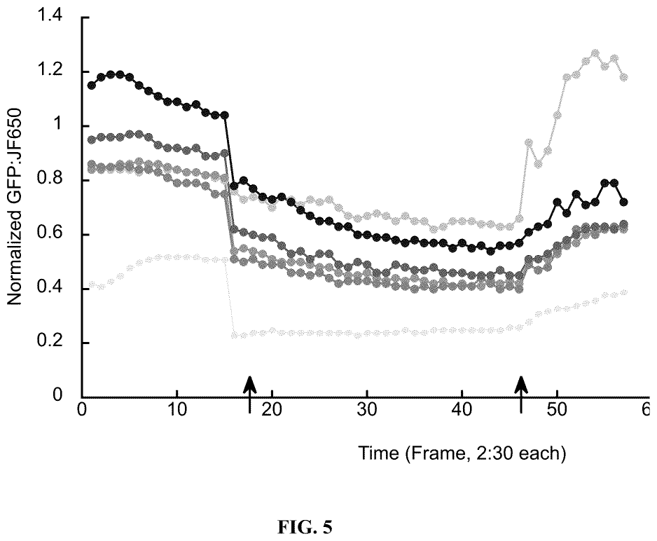

To confirm that iG6PSnFRs report changes in physiologically relevant concentrations of G6P, we transiently transfected HeLa cells with plasmids encoding the 109D9 or 66C5 iG6PSnFRs fused to HaloTag and targeted to the cytosol. In parallel, we also transfected HeLa cells with the glucose sensor, iGlucoSnFR2, fused to HaloTag. After cells were transfected and given 18 hours to recover and express recombinant sensor proteins, we added HaloTagLigand-JFX650 to a concentration of 100 μM in Mammalian Cell Imaging buffer (20 mM HEPES pH 7.5, 120 mM NaCl, 2.5 mM KCl, 2 mM CaCl₂), 2 mM MgCl₂) containing 1 mM glucose. We imaged these cells in a Cytation multi-well plate reader and imaged both the GFP channel to detect G6P and the CY5 channel to detect HaloTag coupled to the far-red fluorophore, JFX650, to normalize for variable protein expression (FIG. 5). We then performed different perturbations that were expected to result in changes in the cytosolic concentrations of G6P.

First, varying the concentration of glucose in the Imaging Buffer results in variable fluorescence from iG6PSnFR expressing cells, i.e., more glucose leads to more G6P being produced. FIG. 5.

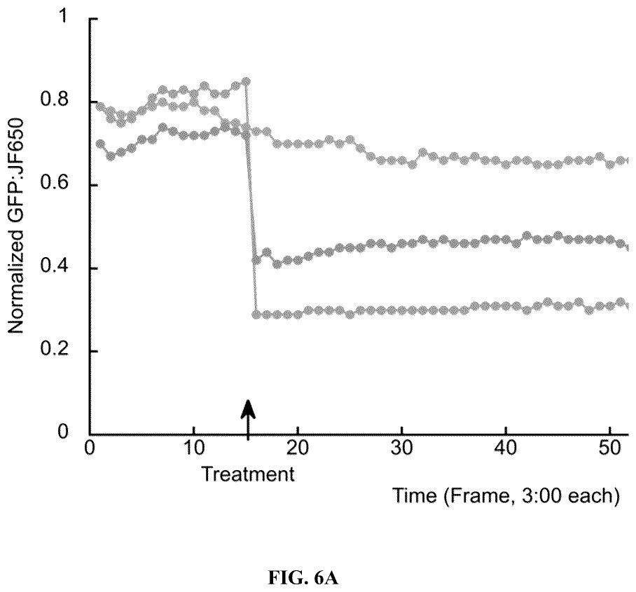

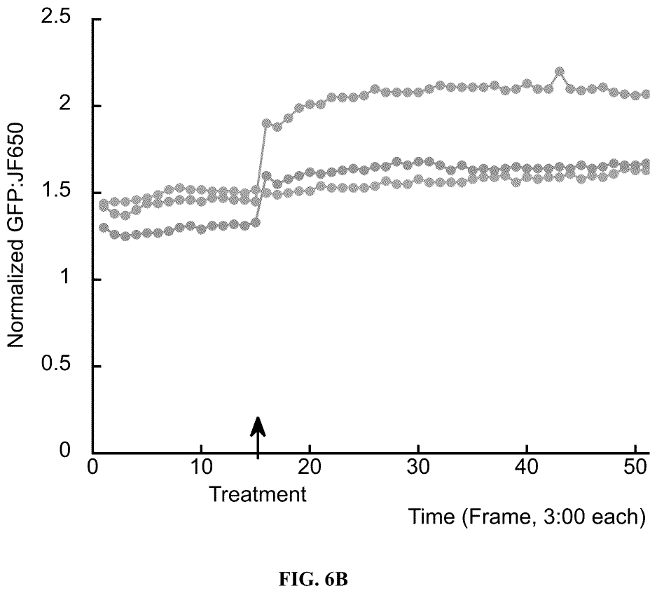

Second, blocking glucose from entering cells by inhibition of the GLUT family of glucose transporters with the drug Glutor resulted in rapid depletion of G6P since G6P stops being produced from glucose yet is still consumed from multiple pathways. iG6PSnFR 109D9 showed a rapid decrease in fluorescence when Glutor was added to the Imaging Buffer, regardless of the concentration of glucose in the Imaging Buffer (FIG. 6A), whereas sensor 66C5 showed the expected inverse response (i.e., fluorescence increases when [G6P] decreases) (FIG. 6B).

Third, blocking G6P production by inhibiting hexokinase with 2-deoxyglucose causes a similar drop in fluorescence for iG6PSnFR variant 109D9 (FIG. 6A) and an increase in fluorescence with variant 66C5 (FIG. 6B).

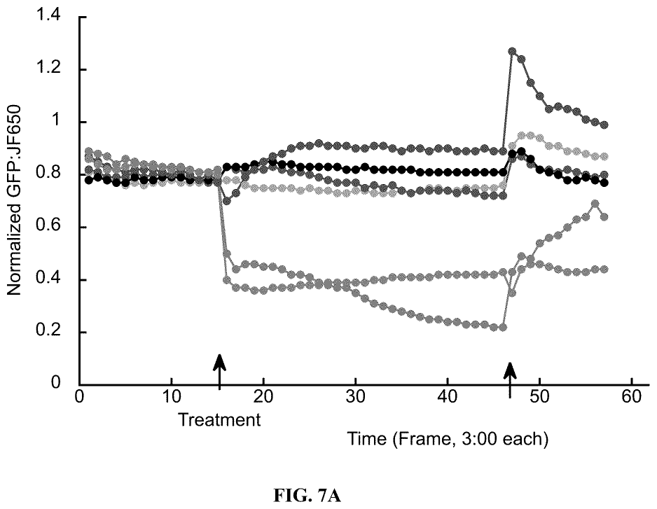

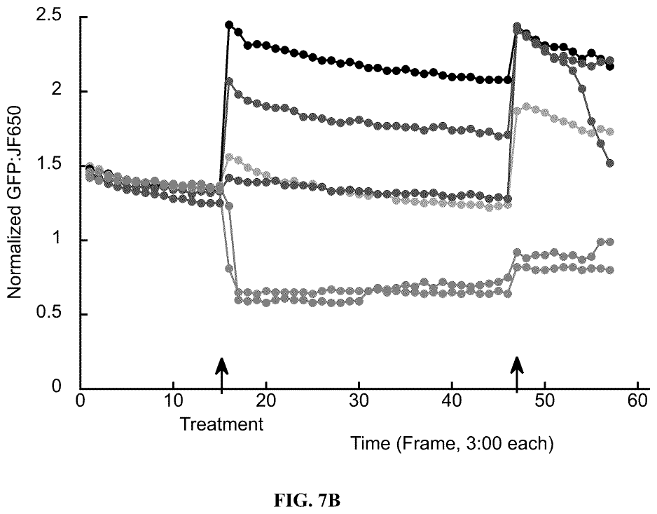

Fourth, iG6PSnFR, in conjunction with iGlucoSnFR2, can be useful for screening or validating drug candidates that modulate cellular metabolism. For example, the drug Lonidamine was thought to be an inhibitor of hexokinase. When HeLa cells expressing iG6PSnFR.109D9 are treated with the GLUT inhibitor Glutor or Lonidamine, fluorescence decreases, indicating a drop in cytosolic G6P (FIG. 7A). When HeLa cells expressing iGlucoSnFR2 are similarly treated, fluorescence also decreases, indicating a drop in cytosolic glucose (FIG. 7B). This implies that Lonidamine is a glucose transport inhibitor, not a hexokinase inhibitor.

### Example 4—Monitoring G6P Dynamics in Neurons

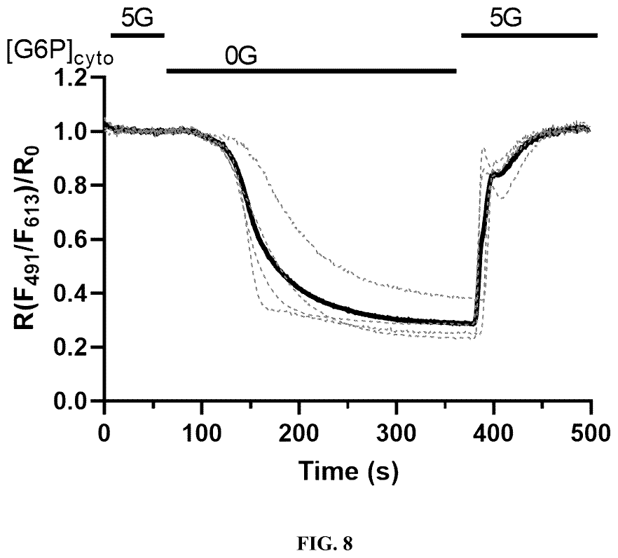

We next tested iG6PSnFR in cultured neurons. Just as with immortalized HeLa cells, removal of glucose from the media of results in a drop in cytosolic G6P, which can be restored by replenishing glucose. See FIG. 8.

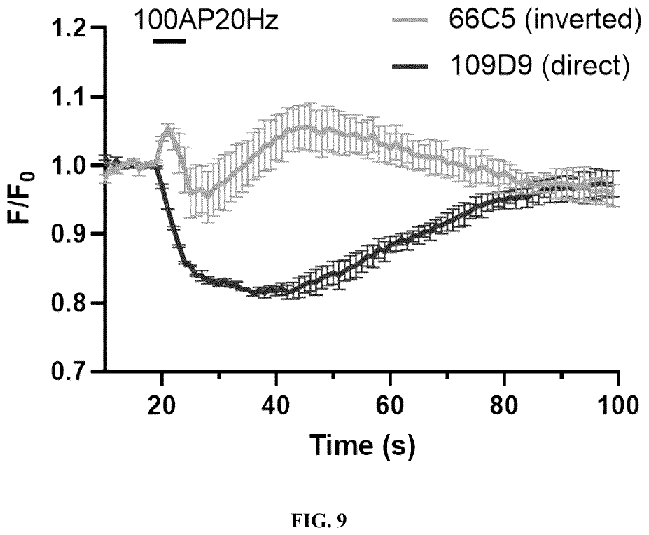

We have previously shown that neurons are highly dependent on glycolysis to provide energy during electrical stimulation. With iG6PSnFR targeted to boutons via synaptophysin, we observe that a train of 100 stimuli reduced G6P pools. The normal sensor and the inverse sensor give complementary responses. Note that the immediate drop in the inverted sensor might be a result of changes in pH. See FIG. 9.

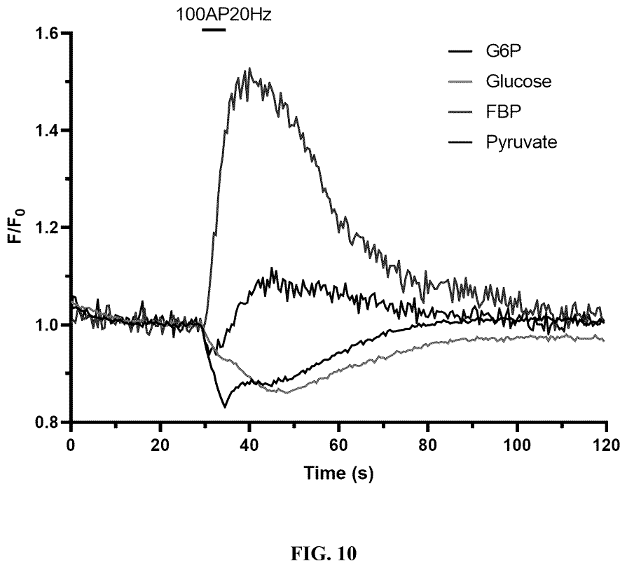

The change in glycolytic metabolite concentrations was further validated by observing changes in four species in parallel experiments using iGlucoSnFR2, iG6PSnFR.109D9, HyLight (which detects fructose-1,6-bisphosphate), and Pegassos (which detects pyruvate). Zooming in on the period of stimulation, we see an immediate rise in FBP and concomitant decrease in G6P. There is a slower, but also immediate decrease in glucose. Once the stimulus is ended, FBP and G6P drift back to their starting equilibrium values, while glucose homeostasis takes longer. Pyruvate increases, but being at the end of the glycolysis, the timing of its increase is significantly delayed. See FIG. 10.

### Example 5—iG6PSnFR Reports Induced Oscillations of Glucose-6-Phosphate in Isolated Pancreatic Islets

Because of its central position at the entry point of glucose metabolism, monitoring G6P in pancreatic islets can provide valuable insight into how β cells sense glucose and regulate insulin release under physiological and disease conditions. To that end, we expressed the 66C5 variant of iG6PSnFR in islets via adenoviral expression. (The fluorescence of the 109D9 variant, which is dim and gets brighter in the presence of G6P was difficult to separate from the fluorescence of NADH, which is present at high levels in islets.). It is well established that when islets are transitioned from low glucose (2 mM) to high glucose (10 mM), a cascade of signaling events is initiated, culminating in the synchronized release of insulin. Rising glycolysis increases ATP concentrations. ATP-sensitive potassium channels close at higher ATP concentrations, leading to depolarization of the plasma membrane. That opens voltage-gated calcium channels.

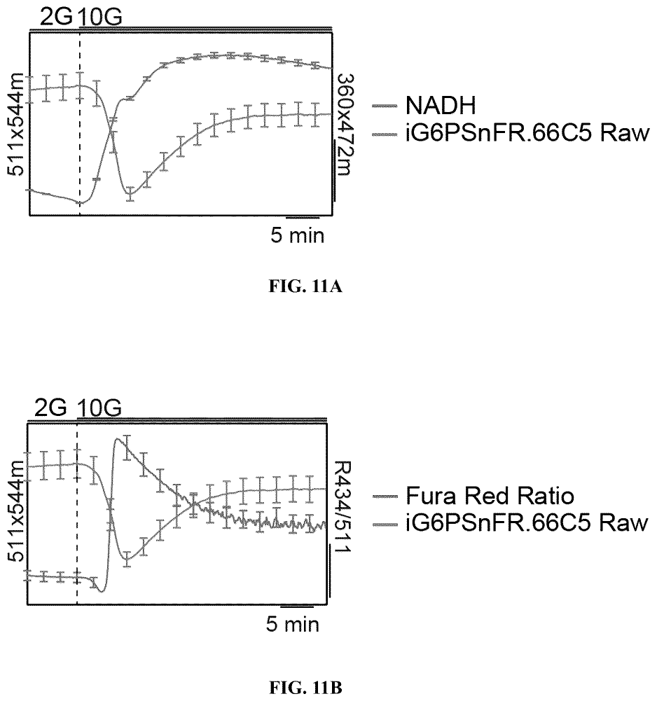

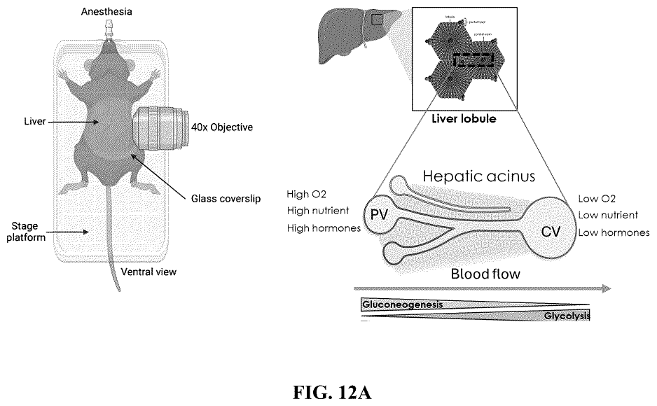

Three channel imaging was used to record these events. The intrinsic fluorescence of NADH was recorded in the blue channel (Ex 340 nm, em 460 nm). It is often used to report glycolysis in cells. When glycolysis is high, NAD+ is reduced to NADH and fluorescence at 460 nm increases. The fluorescence of the 66C5 variant of iG6PSnFR was recorded in the green channel (Ex 475, em 515 nm) to report changes in G6P concentration (FIG. 11A). We also used FuraRed, a calcium sensitive fluorescent reporter that has a broad excitation spectrum and emits at 640 nm (FIG. 11B). Observing these three channels in individual islets that were transitioned from 2 mM glucose to 10 mm glucose, the NADH rises immediately, followed by a sharp transition with calcium, and then an increase in G6P as indicated by a drop in the fluorescence of the 66C5 inverse sensor.

### Example 6—iG6PSnFR Reports Differences in Steady State G6P Concentrations in Glycolytic and Gluconeogenic Hepatocytes in an Intravital Model

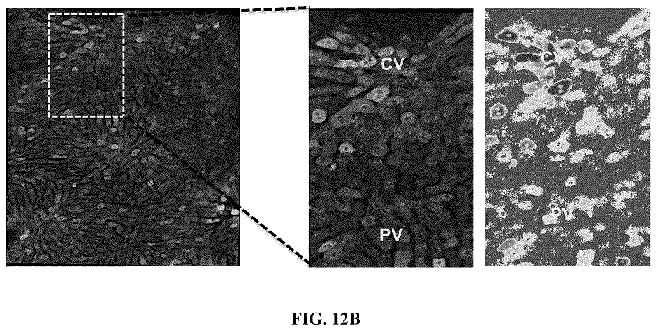

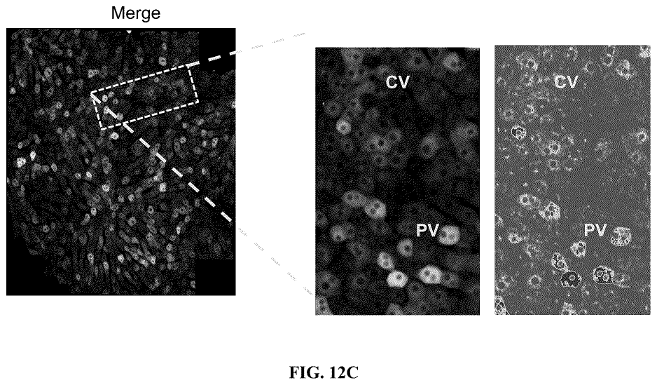

Finally, either 109D9 or 66C5 variants of iG6PSnFR tagged with mIRFP670nano3 via AAV was expressed in the liver of mice. In the liver, there is a portal vein that carries oxygen-rich blood. Cells around that get their energy from oxidative phosphorylation. There is also a Central Vein that has less oxygen and thus hepatocytes near that get their energy from glycolysis. iG6PSnFR shows higher concentrations of glucose-6-phosphate near the Central Vein than by the Portal Vein indicating that glycolytic cells maintain a higher pool of glucose-6-phosphate than gluconeogenic cells. See FIG. 12.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A glucose-6-phosphate (G6P)-detecting fluorescence-based sensor having at least 95% sequence identity to SEQ ID NO: 1 or SEQ ID NO: 2.

2. The G6P-detecting fluorescence-based sensor of claim 1, having at least 96% sequence identity to SEQ ID NO: 1 or SEQ ID NO: 2.

3. The G6P-detecting fluorescence-based sensor of claim 1, having at least 97% sequence identity to SEQ ID NO: 1 or SEQ ID NO: 2.

4. The G6P-detecting fluorescence-based sensor of claim 1, having at least 98% sequence identity to SEQ ID NO: 1 or SEQ ID NO: 2.

5. The G6P-detecting fluorescence-based sensor of claim 1, having at least 99% sequence identity to SEQ ID NO: 1 or SEQ ID NO: 2.

6. The G6P-detecting fluorescence-based sensor of claim 1, having 100% sequence identity to SEQ ID NO: 1 or SEQ ID NO: 2.

7. A nucleic acid encoding the G6P-detecting fluorescence-based sensor of claim 1.

8. A vector comprising the nucleic acid of claim 7.

9. A cell comprising the nucleic acid of claim 7.

10. A cell comprising the vector of claim 8.

11. A method of determining the amount of glucose-6-phosphate (G6P) in a cell, comprising:
providing a cell comprising the G6P-detecting fluorescence-based sensor of claim 1, and
determining the amount of fluorescence emitted from the sensor.

12. The method of claim 11, further comprising exposing the cell to G6P, an inhibitor of G6P, or another biochemical stimuli.

13. The method of claim 11, wherein the cell expresses the nucleic acid of claim 7.

14. The method of claim 11, wherein the cell comprises the vector of claim 8.

15. The method of claim 11, wherein the cell is in vivo.

16. The method of claim 11, wherein the cell is from an organism that suffers from a metabolic disorder.

17. An article of manufacture, comprising the G6P-detecting fluorescence-based sensor of claim 1.

18. The article of manufacture of claim 17, further comprising G6P.

19. The article of manufacture of claim 17, further comprising a G6P inhibitor.

20. The article of manufacture of claim 17, further comprising a biochemical stimuli.
